(19) United States
(12) Patent Application Publication
(10) Pub. No.: US 2019/0160134 A1
(43) Pub. Date: May 30, 2019

(54) Formulations for Cosmetic and Dermatological Applications

(71) Applicant: MUNA ABDULRAHMAN MOHAMED ABDULRAHMAN ALHAMMADI, Abu Dhabi (AE)

(72) Inventor: MUNA ABDULRAHMAN MOHAMED ABDULRAHMAN ALHAMMADI, Abu Dhabi (AE)

(21) Appl. No.: 15/824,190

(22) Filed: Nov. 28, 2017

(51) Int. Cl. A61K 36/889; A61K 36/185; A61K 35/644; A61K 31/355; A61K 36/324; A61K 36/14; A61K 36/53; A61K 8/9789; A61K 8/9794; A61K 8/9761; A61K 8/92; A61K 8/67; A61Q 19/00

(52) U.S. Cl. CPC A61K 36/889; A61K 36/185; A61K 35/644; A61K 31/355; A61K 36/324; A61K 36/14; A61K 2800/5922; A61K 8/9789; A61K 8/9794; A61K 8/9761; A61K 8/925; A61K 8/678; A61Q 19/00; A61K 36/53

(57) ABSTRACT

Disclosed are cosmetic and dermatological formulations which contain all natural ingredients, and particularly plant based ingredients, while excluding water, unnecessary fillers, artificial fragrances and harmful preservatives which might be irritants to the skin. The natural plant based formulations presented herein can be used for treating and/or alleviating skin disorders including dermatitis, eczema, rough skin, cracking and itchy skin. The formulations incorporate a mixture of plant based ingredients including Butyrospermum parkii (shea butter), Cocos nucifera (virgin coconut oil), Vera alba (beeswax), essential oils and d-alpha tocophera (vitamin E).

### BACKGROUND

### Field of Invention

The present invention relates to formulations for cosmetic and dermatological applications. More specifically the present invention relates to natural formulations which are useful in the treatment of various skin conditions, including dermatitis, eczema, rough skin, and cracked or itchy skin.

### Description of Related Art

Cosmetic and dermatological products and medicines, which enhance the appearance of skin, and alleviate commonly occurring skin conditions are increasingly in demand. Consumers are interested in mitigating or delaying the symptoms which can often be present with persistent skin conditions, such as dermatitis, eczema, rough skin, and cracked or itchy skin. The symptoms can include redness of the skin, scaling and dryness of the skin, rough or highly porous texture and cracked or itchy skin patches.

Seeking solutions to these common skin conditions can be very costly for a consumer and sometimes pose risk to a consumer, as often the first line of defense that is recommended by a dermatologist may be prescription formulations which include harsh chemicals, fillers and preservatives which may pose other risks to the skin, or alternatively a consumer is sometimes often undergoing expensive medical therapeutic treatments which also can cause irritation to the skin and do not provide a safe and natural remedy.

It is largely believed that a defects in the skin barrier can increase an individual's susceptibility to microbes, colonisation, as well as penetration of allergens and toxins into the skin. The skin is a key portal of entry for relevant allergens and irritants that can result in various skin diseases described above. Repairing the epidermal barrier defect may be effective approach to preventing and/or treating skin diseases.

In order to maintain normal skin functions, active ingredients derived from plants and plant seeds have commonly been employed in topical compositions for a myriad of medicinal, therapeutic and cosmetic purposes. Such actives can be obtained from various parts of a plant such as seeds, needles, leaves, roots, bark, cones, stems, rhizomes, callus cells, protoplasts, organs and organ systems, and meristems. Active ingredients are incorporated in such compositions in a variety of forms. Such forms include a pure or semi-pure component, a solid or liquid extract or derivative, or a solid plant matter.

It is therefore an object of the present invention to provide a cosmetic and dermatological formulations which alleviate all the current drawbacks of the products available on the market currently.

More specifically, it is an object of the present invention to provide formulations with natural plants extracts which provide immediate hydration of the skin and prevent water loss, boost skin cell regeneration, form a barrier over the skin, and protect it from external irritants while strengthening the skin structure.

It is a further object of the present invention to provide formulations which do not contain any water, unnecessary fillers, artificial fragrances or chemical ingredients, or harmful preservatives.

It is an additional object of this invention to provide formulations with natural ingredients which have inherent vitamins, minerals, antioxidants, antibacterial, antiviral and anti-inflammatory properties.

Lastly, the formulations of the present invention will provide a safe, effective and novel means to treat, prevent, reduce, inhibit, and/or improve the dermatological symptoms of the above discussed skin disorders. As described herein, novel and beneficial compositions, as well as their mode of action, cosmetic and therapeutic treatments, as well as for personal care products for the skin, are provided by the present invention.

### SUMMARY OF THE INVENTION

The novel cosmetic and dermatological formulations of the present invention contain all natural ingredients, and particularly plant based ingredients, while excluding water, unnecessary fillers, artificial fragrances or other chemical ingredients and harmful preservatives which might be irritants to the skin. The natural plant based formulations presented herein can be used for treating and/or alleviating skin disorders including dermatitis, eczema, rough skin, cracking and itchy skin. The natural ingredients selected for these formulations all have a specific purpose and were chosen based on their potency and well-proven healing properties.

Study of the effects produced by the use of the cosmetic and dermatological formulations of the present invention have shown that the condition of the skin is substantially and improved through the use of these formulations.

In one embodiment of the present invention a cosmetic or dermatological formulation is presented which incorporates the following ingredients:
Butyrospermum parkii (shea butter);
Cocos nucifera (virgin coconut oil);
Vera alba (beeswax);
at least one essential oil; and
d-alpha tocophera (vitamin E).

The essential oils to be incorporated in the present formulations can be selected from the group Boswellia carteri (frankincense essential oil), Juniperus virginiana (cedarwood essential oil), and Lavandula officinalis (lavender essential oil). Depending on the formulation, the potency required and its particular use, in one embodiment all three of the essential oils incorporated present in the formulation, or alternatively they can be present singularly or in any combination with each other.

In a preferred embodiment of the present invention, the ingredients of the formulations are present in the following weight percent amounts:
Butyrospermum parkii (Shea butter) is present at about 31.5 wt %;
Cocos nucifera (virgin coconut oil) is present at about 35.5 wt %;
Vera alba (beeswax) is present at about 31.5 wt %;
at least one essential oil is present at about 0.95 wt %; and
d-alpha tocophera (vitamin E liquid) is present at about 0.55 wt %.

In this particular preferred embodiment the essential oils are present in the following weight percent amounts:
Boswellia carteri (frankincense essential oil) present at about 0.40 wt %;
Juniperus virginiana (cedarwood essential oil) present at about 0.275 wt %; and
Lavandula officinalis (lavender essential oil) present at about 0.275 wt %.

In more general embodiment, the essential oils component of the formulations can be incorporated at a weight percent range from 0.25-50 wt %. The various percentages of the individual oils can vary within this range. The incorporation of the essential oils and their respective percentages, including the percentage of the essential oils in total, will vary on the efficacy required, the potency desired, and the particular use and target skin condition of the formulation. Generally, the higher the essential oil component is within the formulation, the more potent the dermatological effect of the formulation on the skin of a user. Therefore, depending on the potency of the formulation, the use guidelines may vary, for example some formulations may be recommended for daily use or twice daily use if they have low essential oil content and are not particularly potent, whereas a higher essential oil content may require less frequent use, for example once daily or once every few days and so on.

In one embodiment of the present invention the Boswellia carteri (francincense essential oil) may incorporate any one of or a combination of Boswellia papyrifera and Boswellia frereana.

In another embodiment, the Juniperus virginiana (cedarwood essential oil) may incorporate any one of or a combination of juniperud ashei, cedrus attantica, C. libani, C. Linanivar, C. deodara, and African cedar.

In a further embodiment, the Lavandula officinalis (lavender essential oil) may incorporate any one of or a combination of L. angustifolia, L. Spica, L. spicata, L. stoechas, L×intermedia, L. officinalis, L. vera, L latifolia, and L dentate.

In one embodiment the cosmetic and dermatological formulation of the present invention can contain the natural plant based ingredients in the following weight percentage amounts:
Butyrospermum parkii (shea butter) is present at a range of 11.00-63.25 wt %;
Cocos nucifera (virgin coconut oil) is present at a range of wt 25.00-77.25%;
Vera alba (beeswax) is present at a range of 11.00-35.00 wt %;
at least one essential oil is present at a range of 0.25-50.00 wt %; and
d-alpha tocophera (vitamin E) is present at a range of 0.50-1.00 wt %.

The essential oils in the formulation can have the following weight percentage amounts:
Boswellia carteri (frankincense essential oil) present at a range of 0.105-21.1 wt %;
Juniperus virginiana (cedarwood essential oil) present at a range of 0.0724-14.47 wt %; and
Lavandula officinalis (lavender essential oil) present at a range of 0.0724-14.47 wt %.

The above summarised formulations aid in maintaining normal skin functions, and the carefully selected natural plants extracts provide immediate hydration of the skin and prevent water loss, in addition to boosting skin cell regeneration, forming a barrier over the skin and protect it from external irritants and strength skin structure.

Other features and advantages of the present invention will become more apparent from the following detailed description of the invention when taken in conjunction with the appended claims.

### DETAILED DESCRIPTION

Disclosed herein are novel cosmetic and dermatological formulations containing all natural plant-based ingredients. The natural formulations presented in the following embodiments can be used for treating and/or alleviating symptoms associated with various skin disorders, including dermatitis, eczema, rough skin, cracking and itchy skin. Contrary to common cosmetic or dermatological products currently on the market, the natural formulations of this invention do not include any added water, unnecessary fillers, artificial fragrances or other chemical ingredients and harmful preservatives which can often exacerbate already inflamed dry or otherwise problematic skin conditions.

It is largely believed that defects in the skin barrier increase the susceptibility to microbes, as well as penetration of unwanted allergens and toxins into the skin. Repairing the epidermal barrier defect is an effective approach to preventing and/or treat various skin diseases.

The selected natural plants extracts of the present invention have been found to provide immediate hydration of the skin and prevent water loss, boost skin cell regeneration, form a barrier over the skin and protect it from external irritants while strengthening the skin structure.

The formulations are based on a moisturising system which adjusts to the specific needs of the skin while the active components are allowed to be released into the skin. Additionally, the formulations disclosed herein have been expertly balanced in a way that the composition themselves have a self-preserving effect. That is to say unnecessary preservatives are not added to these natural plant-based formulations.

The organic composition used in this formula, are preferably cold pressed to preserve their inherent vitamins, minerals, antioxidants, in addition to their antibacterial, antiviral and anti-inflammatory properties.

In one embodiment of the present invention a cosmetic or dermatological formulation is presented which incorporates the following ingredients:
Butyrospermum parkii (shea butter);
Cocos nucifera (virgin coconut oil);
Vera alba (beeswax);
at least one essential oil; and
d-alpha tocophera (vitamin E).

The essential oils to be incorporated in the present formulations can be selected from the group Boswellia carteri (frankincense essential oil), Juniperus virginiana (cedarwood essential oil), and Lavandula officinalis (lavender essential oil). Depending on the formulation, the potency required and its particular use, in one embodiment all three of the essential oils incorporated present in the formulation, or alternatively they can be present singularly or in any combination with each other.

In a preferred embodiment of the present invention, the ingredients of the formulations are present in the following weight percent amounts:
Butyrospermum parkii (Shea butter) is present at about 31.5 wt %;
Cocos nucifera (virgin coconut oil) is present at about 35.5 wt %;
Vera alba (beeswax) is present at about 31.5 wt %;
at least one essential oil is present at about 0.95 wt %; and
d-alpha tocophera (vitamin E liquid) is present at about 0.55 wt %.

In this particular preferred embodiment the essential oils are present in the following weight percent amounts:
Boswellia carteri (frankincense essential oil) present at about 0.40 wt %;
Juniperus virginiana (cedarwood essential oil) present at about 0.275 wt %; and
Lavandula officinalis (lavender essential oil) present at about 0.275 wt %.

In more general embodiment, the essential oils component of the formulations can be incorporated at a weight percent range from 0.25-50 wt %, or 1-40 wt %, or 10-30 wt %, or 15-25 wt %. The various percentages of the individual oils can vary within this range. The incorporation of the essential oils and their respective percentages, including the percentage of the essential oils in total, will vary on the efficacy required, the potency desired, and the particular use and target skin condition of the formulation. Generally, the higher the essential oil component is within the formulation, the more potent the dermatological effect of the formulation on the skin of a user. Therefore, depending on the potency of the formulation, the use guidelines may vary, for example some formulations may be recommended for daily use or twice daily use if they have low essential oil content and are not particularly potent, whereas a higher essential oil content may require less frequent use, for example once daily or once every few days and so on.

In one embodiment of the present invention the Boswellia carteri (francincense essential oil) may incorporate any one of or a combination of Boswellia papyrifera and Boswellia frereana.

In another embodiment, the Juniperus virginiana (cedarwood essential oil) may incorporate any one of or a combination of juniperud ashei, cedrus attantica, C. libani, C. Linanivar, C. deodara, and African cedar.

In a further embodiment, the Lavandula officinalis (lavender essential oil) may incorporate any one of or a combination of L. angustifolia, L. Spica, L. spicata, L. stoechas, L×intermedia, L. officinalis, L. vera, L latifolia, and L dentate.

In one embodiment the cosmetic and dermatological formulation of the present invention can contain the natural plant based ingredients in the following weight percentage amounts:
Butyrospermum parkii (shea butter) is present at a range of 11.00-63.25 wt %, or 15.00-50.00 wt %, or 25.00-45.00 wt %, or 30.00-40.00 wt %;
Cocos nucifera (virgin coconut oil) is present at a range of 25.00-77.25 wt %, or 30.00-65.00 wt %, or 35.00-55.00 wt %, or 40.00-50.00 wt %;
Vera alba (beeswax) is present at a range of 11.00-35.00 wt %, or 15.00-33.00 wt %, or 18.00-30.00 wt %, or 20.00-25.00 wt %;
at least one essential oil is present at a range of 0.25-50.00 wt %, or 1.00-40.00 wt %, or 10.00-30.00 wt %, or 15.00-25.00 wt %; and
d-alpha tocophera (vitamin E) is present at a range of 0.50-1.00 wt %, or 0.60-0.90 wt %, or 0.70-0.80 wt %.

The essential oils in the formulation can have the following weight percentage amounts:
Boswellia carteri (frankincense essential oil) present at a range of 0.105-21.1 wt %, or 1.00-18.00 wt %, or; 5.00-15.00 wt %, or 10.00-12.00 wt %;
Juniperus virginiana (cedarwood essential oil) present at a range of 0.0724-14.47 wt %, or 0.5-12.00 wt %, 1.00-10.00 wt %, 3.00-8.00 wt %; and
Lavandula officinalis (lavender essential oil) present at a range of 0.0724-14.47 wt %, or 0.5-12.00 wt %, 1.00-10.00 wt %, 3.00-8.00 wt %.

### Example Formulation

In the following example, a preferred formulation of the present invention will be disclosed. In the preferred embodiment the current cosmetic and dermatological formulations are mixed according to the following weights. The below mixture results in about 4 containers filled with about 4 ounces (oz) of the mixture.

Butyrospermum parkii (Shea butter) organic premium, grade A, unrefined—4.0 oz
Cocos nucifera (virgin coconut oil)—4.5 oz
Vera alba (beeswax) USDA organic 100% pure grade A—4.0 oz
Boswellia carteri (frankincense essential oil)—25 drops
Juniperus virginiana (cedarwood essential oil)—20 drops
Lavandula officinalis (lavender essential oil)—20 drops
d-alpha tocophera (natural vitamin E liquid)—40 drops

The above formulation is made by mixing Shea butter, beeswax and coconut oil in sterile mixing glass. A large pot is filled with 1 or 2 inches (2.5-5 cm) of water, placed on the heat and heated on high until the water reaches a steady, rolling boil. Then, mixing glass is set inside the bottom half, and the heat is lowered (ensuring also that no water can get inside the mixing glass). The mixture start to melt slowly, the coconut oil starts melting between (25-26 degrees C.), followed by the Shea butter (40-45 degrees C.), and last the beeswax (63-64 degrees C.). The mixture should not exceed 71 degrees C.

Once the mixture has finished melting it is taken off from heat, essential oils are added after the mixture is cooled for 2 minutes. The mixture is mix while adding the essential oils and stirring every two minutes for about 20-25 minutes. The mixture is then left to cool at room temperature for about one hour. Lastly the cooled mixture and final product is then put in packaging tubes or glass containers.

The above detailed formulations have been shown to aid in maintaining normal skin functions, and the carefully selected natural plants extracts provide immediate hydration of the skin and prevent water loss, in addition to boosting skin cell regeneration, forming a barrier over the skin and protect it from external irritants and strength skin structure.

Study of the effects produced by the use of the cosmetic and dermatological formulations of the present invention have shown that the condition of the skin is substantially and improved through the use of these formulations. Various benefits and therapeutic pathways of each of the ingredients are detailed below.

Butyrospermum parkii (Shea Butter)

Shea butter is known to be rich in vitamins E and A, in addition to fatty acids and plant sterols, like oleic, stearic, palmitic and linolenic acids. Shea butter has several anti-inflammatory agents, including derivatives of cinnamic acid which has great healing potential for the skin. According to the American Shea Butter Institute, the moisturisers in Shea butter are the same ones that are produced by the skin's sebaceous glands, making it one of the best matches for dry skin.

Although Shea butter is utilized in preferred embodiments in the formulations of the present invention, other comparable ingredients can also be used as a substitute to shea butter, such as for example are mango butter, cocoa butter, kokum butter, cupuacu butter, ucuuba butter, tamanu butter, tucuma butter, murumuru butter, illipe nut butter, and sal butter.

Cocos nucifera (Virgin Coconut Oil)

Coconut oil consists of more than 90% of saturated fats, along with traces of a few unsaturated fatty acids, such as monounsaturated fatty acids and polyunsaturated fatty acids.

The saturated fatty acids are mostly medium chain triglycerides, which are known to assimilate well in the body's systems. Lauric acid is the chief contributor to coconut oil, representing more than 40% of the total, followed by capric acid, caprylic acid, myristic acid and palmitic. Lauric acid is helpful in dealing with viruses and diseases. Capric acid reacts with certain enzymes secreted by other bacteria, which subsequently convert it into a powerful antimicrobial agent, monocaprin. Caprylic acid, caproic acid and myristic acid are rich in antimicrobial and antifungal properties as well. Coconut oil also contains poly-phenols such as Gallic acid, which is also known as phenolic acid. These polyphenols are responsible for the fragrance and the taste of coconut oil and Virgin Coconut Oil is rich in these polyphenols. Derivatives of fatty acid like Betaines, ethanolamide, ethoxylates, fatty esters, fatty polysorbates, monoglycerides and polyol esters are also present.

The medium-chain fatty acids, specifically lauric acid, capric acid and caprylic acid, are known to boost the immune system and eliminate bacteria in topical applications. The active ingredients in coconut oil, such as vitamin E and K, as well as volatile organic acids, help to stimulate the regrowth of new cells and prevent the negative effects of free radicals and oxidative stress, which in turns helps the skin combat the normal signs of ageing.

Coconut oil penetrates or absorbs into the skin and thus facilitate the absorption of other oils as well (such as essential oils) and herbal extracts through the skin when they are mixed therein. Coconut oil is easily absorbed through the skin's pores and thus is used as carrier oil. Furthermore, being one of the most stable oils, it does not go rancid, nor does it let the other oils or herbal extracts spoil inside of it. It also does not alter the properties of the oils and herbs mixed within. Additionally, it protects the herbs and oils from microbial or fungal interactions. Coconut oil helps in treating various skin problems, including psoriasis, dermatitis, eczema and other skin infections. For that exact reason, coconut oil forms the base ingredient of various body care products like soaps, lotions, and creams that are used for skin care.

Although coconut oil is utilized in preferred embodiments in the formulations of the present invention, other comparable ingredients can also be used as a substitute to coconut oil such as for example apricot kernel, argan, avocado, evening primrose, hazelnut, jojoba, macadamia, neem, tamanu, sesame, rosehip, grapeseed, pomegranate, olive, sweet almond, caster, babassu oil and maracuja oil.

Vera alba (Beeswax)

Beeswax has Vitamin A, which improves hydration to the skin and promotes cell regeneration. A wide variety of cosmetics products utilize beeswax because not only is it natural and non-toxic, it provides benefits to the skin and to various types of formulas. It's a thickening agent for instance, which means that it can help thicken creams to make them easier to use and more spreadable on the skin. It also forms a protective barrier that helps protect the skin from environmental assaults, while also holding in moisture and reducing dryness.

Unlike ingredients made from petroleum, however, beeswax does not suffocate the skin, and will not clog pores. It has humectants properties, which means it attracts water molecules, helping to keep skin hydrated over time. Beeswax is a good source of vitamin A, which helps exfoliation and can rejuvenate skin. It also has a natural honey fragrance, and a pleasant, light aroma. Most importantly, Beeswax is anti-allergenic which means it can be easily tolerated by individuals which might have sensitive or reactive skin.

Although beeswax is utilized in preferred embodiments in the formulations of the present invention, other comparable ingredients can also be used as a substitute to beeswax such as emulsifying wax and carnauba wax.

Boswellia carteri (Frankincense Essential Oil)

The main components of frankincense essential oil are ketonic alcohol (olibanol), resinous matters (30-60%) and terpenes such as a- and p-pinene, camphene, dipentene and phellandrene. It also contains alpha pinene, actanol, bornyl acetate, linalool, octyl acetate, incensole and incensyl acetate.

The monoterpenes and sesquiterpenes are the most valuable elements of frankincense oil.

The sesquiterpenes can go beyond the blood-brain barrier and help simulate the limbic system of your brain, as well as your hypothalamus, and pineal and pituitary glands.

Frankincense oil contains a complex mixture of monoterpenes such as alpha-pinene, Limonene, alpha-Thujene, and beta-Pinene. This intricate mixture of components works together to make an oil with many uses and benefits. The unique chemical constituents contribute to frankincense essential oil's renewing effects when applied topically and ability to support diverse cells in the body when taken internally. The monoterpenes also contribute to the aromatic benefits of Frankincense oil, including its ability to promote relaxation and a sense of focus.

Juniperus virginiana (Cedarwood Essential Oil)

Cedarwood is a plant native to cold climates and is normally found at high altitudes. The chief components of Cedarwood essential oil are Alpha Cedrene, Beta Cedrene, Cedrol, Widdrol, Thujopsene and a group of Sesquiterpenes, which contribute a greatly to its medicinal value and health benefits.

The health benefits of Cedarwood essential oil can be attributed to its properties as an antiseborrhoeic, antiseptic, antispasmodic, tonic, astringent, diuretic, emenagogue, expectorant, insecticidal, sedative and fungicidal substance. Cedarwood essential oil prevents wounds from becoming septic as well as protecting them from tetanus germs. It can safely be applied externally on wounds as an antiseptic, and it is even used as an ingredient in herbal antiseptic creams. By defending the body against toxins, the Cedarwood essential oil relieves the white blood cells and immune system of the extra stress, which allows them to better protect internal functions.

Lavandula officinalis (Lavender Essential Oil)

Lavender essential oil inhibits the bacteria that cause initial dermal infections and helps to regulate some of the over-excretion of sebum by hormonal manipulation, and can reduce the signs of scarring.

The health benefits of lavender oil for the skin can be attributed to its antiseptic and anti-fungal properties. It is useful in the treatment of various skin disorders such as acne, wrinkles, psoriasis, and other inflammatory conditions. It is commonly used to speed up the healing process of wounds, cuts, burns, and sunburns because it improves the formation of scar tissues. It has ability to eliminate nervous tension, relieve pain, disinfect the scalp and skin, enhance blood circulation and treat respiratory problems.

D-Alpha Tocophera (Vitamin E)

Vitamin E is an antioxidant vitamin found naturally in nuts, seeds, whole grains, vegetable oils, vegetables and some meat and dairy products. D-alpha-tocopherol is the most active form of vitamin E. It helps to protect cells against oxidative damage that contributes to some chronic diseases and premature aging.

There is evidence that the natural form of alpha-tocopherol is better absorbed and has more biological activity than synthetic vitamin E forms. Due to its antioxidant properties, Vitamin E also acts as a natural preservative to extend the life of cosmetic or dermatological products, preventing them from oxidizing and going rancid (and allowing the present formulations to eliminate any use of additional preservatives). In this way vitamin E acts as a potent active ingredient and as a preservative ingredient.

Vitamin E has also been shown to significantly reduce the appearance of stretch marks on the skin and it can even help scars to gradually fade away. Furthermore, scientific studies have shown vitamin E can help decrease the effects of more serious skin conditions such as those of concern of the present invention, including psoriasis and erythema, and it also may help in reducing the risk of skin cancer. In the present invention, Vitamin E is preferably incorporated in liquid form.

The formulations of the present inventions can be used both in dermatological topical applications, compositions, creams, lotions, spreads and so on, and also incorporated in cosmetic products, including make-ups, tinted moisturizing creams, liquid foundations, sunscreen lotions and other types of cosmetic products.

More specifically the formulations disclosed herein can be incorporated with cosmetic or dermatological products such as anti-ageing creams or lotions, anti-wrinkle cream or lotion, eye creams, shaving creams, bath melts or bath lotions, soap bars, lip balms and so on.

Furthermore the formulation of the essential oils can be also used and incorporated as body sprays for the skin, for hair, for the face, and also as sprays for pillows, furniture, room sprays and perfumes.

Preferred embodiments of this invention have been described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein.

1. A cosmetic or dermatological formulation comprising:
Butyrospermum parkii (shea butter);
Cocos nucifera (virgin coconut oil);
Vera alba (beeswax);
at least one essential oil; and
d-alpha tocophera (vitamin E).

2. The cosmetic or dermatological formulation of claim 1, wherein the at least one essential oil is selected from the group Boswellia carteri (frankincense essential oil), Juniperus virginiana (cedarwood essential oil), and Lavandula officinalis (lavender essential oil).

3. The cosmetic or dermatological formulation of claim 1, wherein the at least one essential oil comprises the following essential oils:
Boswellia carteri (frankincense essential oil);
Juniperus virginiana (cedarwood essential oil); and
Lavandula officinalis (lavender essential oil).

4. The cosmetic or dermatological formulation of claim 1, wherein:
Butyrospermum parkii (Shea butter) is present at about 31.5 wt %;
Cocos nucifera (virgin coconut oil) is present at about 35.5 wt %;
Vera alba (beeswax) is present at about 31.5 wt %;
at least one essential oil is present at about 0.95 wt %; and
d-alpha tocophera (vitamin E) is present at about 0.55 wt %.

5. The cosmetic or dermatological formulation of claim 4, wherein the at least one essential oil comprises:
Boswellia carteri (frankincense essential oil) present at about 0.40 wt %;
Juniperus virginiana (cedarwood essential oil) present at about 0.275 wt %; and
Lavandula officinalis (lavender essential oil) present at about 0.275 wt %.

6. The cosmetic or dermatological formulation of claim 1, wherein the at least one essential oils are present at about 0.25-50 wt %.

7. The cosmetic or dermatological formulation of claim 3, wherein the Boswellia carteri (francincense essential oil) comprises any one of or a combination of Boswellia papyrifera and Boswellia frereana.

8. The cosmetic or dermatological formulation of claim 3, wherein the Juniperus virginiana (cedarwood essential oil) comprises any one of or a combination of juniperud ashei, cedrus attantica, C. libani, C. Linanivar, C. deodara, and African cedar.

9. The cosmetic or dermatological formulation of claim 3, wherein the Lavandula officinalis (lavender essential oil) comprises any one of or a combination of L. angustifolia, L. Spica, L. spicata, L. stoechas, L×intermedia, L. officinalis, L. vera, L latifolia, and L. dentate.

10. The cosmetic or dermatological formulation of claim 1, wherein:
Butyrospermum parkii (shea butter) is present at a range of 11.00-63.25 wt %;
Cocos nucifera (virgin coconut oil) is present at a range of wt 25.00-77.25%;
Vera alba (beeswax) is present at a range of 11.00-35.00 wt %;
at least one essential oil is present at a range of 0.25-50.00 wt %; and
d-alpha tocophera (vitamin E) is present at a range of 0.50-1.00 wt %.

11. The cosmetic or dermatological formulation of claim 10, wherein:
Boswellia carteri (frankincense essential oil) present at a range of 0.105-21.1 wt %;
Juniperus virginiana (cedarwood essential oil) present at a range of 0.0724-14.47 wt %; and
Lavandula officinalis (lavender essential oil) present at a range of 0.0724-14.47 wt %.
